(19) United States
(12) Patent Application Publication
(10) Pub. No.: US 2022/0245400 A1
(43) Pub. Date: Aug. 4, 2022

(54) AUTONOMOUS SEGMENTATION OF THREE-DIMENSIONAL NERVOUS SYSTEM STRUCTURES FROM MEDICAL IMAGES

(71) Applicant: HOLO SURGICAL INC., Chicago, IL (US)

(72) Inventors: Krzysztof B. SIEMIONOW, Chicago, IL (US); Cristian J. LUCIANO, Chicago, IL (US); Dominik GAWEL, Warsaw (PL); Edwing Isaac MEJIA OROZCO, Warsaw (PL); Michal TRZMIEL, Warsaw (PL)

(73) Assignee: HOLO SURGICAL INC., Chicago, IL (US)

(21) Appl. No.: 17/708,907

(22) Filed: Mar. 30, 2022

Related U.S. Application Data
(63) Continuation of application No. 16/677,707, filed on Nov. 8, 2019.

(30) Foreign Application Priority Data
Nov. 8, 2018 (EP) 18205207.6

(51) Int. Cl. G06K 9/62; G06T 7/62; G16H 30/40; G06N 20/00; A61B 5/00; G06T 7/00; G06V 10/22; G06V 20/64

(52) U.S. Cl. CPC G06K 9/6256; G06T 7/62; G16H 30/40; G06N 20/00; A61B 5/4058; A61B 5/4566; G06V 2201/034; G06V 10/22; G06V 20/653; G06T 2207/10088; G06T 2207/30008; G06V 2201/033; G06T 7/0014

(57) ABSTRACT

A method for autonomous segmentation of three-dimensional nervous system structures from raw medical images, the method including: receiving a 3D scan volume with a set of medical scan images of a region of the anatomy; autonomously processing the set of medical scan images to perform segmentation of a bony structure of the anatomy to obtain bony structure segmentation data; autonomously processing a subsection of the 3D scan volume as a 3D region of interest by combining the raw medical scan images and the bony structure segmentation data, wherein the 3D ROI contains a subvolume of the bony structure with a portion of surrounding tissues, including the nervous system structure; autonomously processing the ROI to determine the 3D shape, location, and size of the nervous system structures by means of a pre-trained convolutional neural network (CNN).

### CROSS-REFERENCE TO RELATED APPLICATIONS

This application is a continuation of U.S. patent application Ser. No. 16/677,707, filed Nov. 8, 2019, entitled “Autonomous Segmentation of Three-Dimensional Nervous System Structures from Medical Images”, which claims benefit of European Application No. 18205207.6, filed Nov. 8, 2018, entitled “Autonomous Segmentation of Three-Dimensional Nervous System Structures from Medical Images”, the entire disclosure of which is incorporated herein by reference.

### TECHNICAL FIELD

The invention generally relates to autonomous segmentation of three-dimensional nervous system structures from medical images of human anatomy, which is useful in particular for the field of computer-assisted surgery, surgical navigation, surgical planning, and medical diagnostics.

### BACKGROUND

Image-guided or computer-assisted surgery is a surgical approach where the surgeon uses tracked surgical instruments in conjunction with preoperative or intraoperative images in order to indirectly guide the procedure. Image-guided surgery can utilize medical images acquired both preoperatively and intraoperatively, for example: from computer tomography (CT) or magnetic resonance imaging scanners.

Specialized computer systems can be used to process the medical images to develop three-dimensional (3D) models of the anatomy fragment subject to the surgery procedure. For this purpose, various machine learning technologies are being developed, such as a convolutional neural network (CNN) that is a class of deep, feed-forward artificial neural networks. CNNs use a variation of multilayer perceptrons designed to require minimal preprocessing.

A PCT patent application WO2017091833 (Arterys) discloses autonomous segmentation of anatomical structures, such as the human heart.

A US patent application US2016328630 (Samsung) discloses an object recognition apparatus and method that can determine an image feature vector of a first image by applying a convolution network to the first image.

In the field of image guided surgery, low quality images may make it difficult to adequately identify key anatomic landmarks, which may in turn lead to decreased accuracy and efficacy of the navigated tools and implants. Furthermore, low quality image datasets may be difficult to use in machine learning applications.

Computer tomography (CT) is a common method for generating a 3D volume of the anatomy. CT scanning works like other x-ray examinations. Very small, controlled amounts of x-ray radiation are passed through the body, and different tissues absorb radiation at different rates. With plain radiology, when special film is exposed to the absorbed x-rays, an image of the inside of the body is captured. With CT, the film is replaced by an array of detectors, which measure the x-ray profile.

The CT scanner contains a rotating gantry that has an x-ray tube mounted on one side and an arc-shaped detector mounted on the opposite side. An x-ray beam is emitted in a fan shape as the rotating frame spins the x-ray tube and detector around the patient. Each time the x-ray tube and detector make a 360° rotation and the x-ray passes through the patient's body, the image of a thin section is acquired. During each rotation, the detector records about 1,000 images (profiles) of the expanded x-ray beam. Each profile is then reconstructed by a dedicated computer into a 3D volume of the section that was scanned. The speed of gantry rotation, along with slice thickness, contributes to the accuracy/usefulness of the final image.

Commonly used intraoperative scanners have a variety of settings that allow for control of radiation dose. In certain scenarios high dose settings may be chosen to ensure adequate visualization of all the anatomical structures. The downside is increased radiation exposure to the patient. The effective doses from diagnostic CT procedures are typically estimated to be in the range of 1 to 10 mSv (millisieverts). This range is not much less than the lowest doses of 5 to 20 mSv estimated to have been received by survivors of the atomic bombs. These survivors, who are estimated to have experienced doses slightly larger than those encountered in CT, have demonstrated a small but increased radiation-related excess relative risk for cancer mortality.

The risk of developing cancer as a result of exposure to radiation depends on the part of the body exposed, the individual's age at exposure, the radiation dose, and the individual's gender. For the purpose of radiation protection, a conservative approach that is generally used is to assume that the risk for adverse health effects from cancer is proportional to the amount of radiation dose absorbed and that there is no amount of radiation that is completely without risk.

Low dose settings should be therefore selected for computer tomography scans whenever possible to minimize radiation exposure and associated risk of cancer development. However, low dose settings may have an impact on the quality of the final image available for the surgeon. This in turn can limit the value of the scan in diagnosis and treatment.

Magnetic resonance imaging (MRI) scanner forms a strong magnetic field around the area to be imaged. In most medical applications, protons (hydrogen atoms) in tissues containing water molecules create a signal that is processed to form an image of the body. First, energy from an oscillating magnetic field temporarily is applied to the patient at the appropriate resonance frequency. The excited hydrogen atoms emit a radio frequency signal, which is measured by a receiving coil. The radio signal may be made to encode position information by varying the main magnetic field using gradient coils. As these coils are rapidly switched on and off, they create the characteristic repetitive noise of an MRI scan. The contrast between different tissues is determined by the rate at which excited atoms return to the equilibrium state. Exogenous contrast agents may be given intravenously, orally, or intra-articularly.

The major components of an MRI scanner are: 1) the main magnet, which polarizes the sample, 2) the shim coils for correcting inhomogeneities in the main magnetic field, 3) the gradient system, which is used to localize the MR signal, and 4) the RF system, which excites the sample and detects the resulting NMR signal. The whole system is controlled by one or more computers.

The most common MR1 strengths are 0.3 T, 1.5 T and 3 T. The “T” stands for Tesla—the unit of measurement for the strength of the magnetic field. The higher the number, the stronger the magnet. The stronger the magnet the higher the image quality. For example, a 0.3 T magnet strength will result in lower quality imaging then a 1.5 T. Low quality images may pose a diagnostic challenge as it may be difficult to identify key anatomical structures or a pathologic process. Low quality images also make it difficult to use the data during computer assisted surgery. Thus, it is important to have the ability to deliver a high quality MR image for the physician.

### SUMMARY OF THE INVENTION

There is a need to develop a system and a method for efficiently segmenting three-dimensional nervous system structures from intraoperative and presurgical medical images in an autonomous manner, i.e. without human intervention in the segmentation process.

One aspect of the invention is a method for autonomous segmentation of three-dimensional nervous system structures from raw medical images, the method comprising: receiving a 3D scan volume comprising a set of medical scan images of a region of the anatomy; autonomously processing the set of medical scan images to perform segmentation of a bony structure of the anatomy to obtain bony structure segmentation data; autonomously processing a subsection of the 3D scan volume as a 3D region of interest by combining the raw medical scan images and the bony structure segmentation data, wherein the 3D ROI contains a subvolume of the bony structure with a portion of surrounding tissues, including the nervous system structure; autonomously processing the ROI to determine the 3D shape, location, and size of the nervous system structures by means of a pre-trained convolutional neural network.

The method may further comprise 3D resizing of the ROI.

The method may further comprise visualizing the output including the segmented nervous system structures.

The method may further comprise detecting collision between an embodiment and/or trajectory of surgical instruments or implants and the segmented nervous system structures.

The nervous-system-structure segmentation CNN may be a fully convolutional neural network model with layer skip connections.

The nervous-system-structures segmentation CNN output may be improved by Select-Attend-Transfer gates.

The nervous-system-structures segmentation CNN output may be improved by Generative Adversarial Networks.

The received medical scan images may be collected from an intraoperative scanner.

The received medical scan images may be collected from a presurgical stationary scanner.

There is also disclosed a computer-implemented system, comprising: at least one non-transitory processor-readable storage medium that stores at least one processor-executable instruction or data; and at least one processor communicably coupled to at least one non-transitory processor-readable storage medium, wherein at least one processor is configured to perform the steps of the method as described herein.

These and other features, aspects and advantages of the invention will become better understood with reference to the following drawings, descriptions and claims.

### BRIEF DESCRIPTION OF DRAWINGS

Various embodiments are herein described, by way of example only, with reference to the accompanying drawings, wherein:

FIG. 1 shows a training procedure in accordance with an embodiment of the invention;

FIG. 2A shows an image used in the system during the procedures, in accordance with an embodiment of the invention;

FIG. 2B shows an image used in the system during the procedures, in accordance with an

embodiment of the invention;

FIG. 2C shows an image used in the system during the procedures, in accordance with an embodiment of the invention;

FIG. 2D shows an example of an automatically defined region of interest used in the process, in accordance with an embodiment of the invention;

FIG. 2E-1 shows three dimensional resizing of a region of interest, in accordance with an embodiment of the invention;

FIG. 2E-2 shows three dimensional resizing of a region of interest, in accordance with an embodiment of the invention;

FIG. 2F shows an example of transformation for data augmentation, in accordance with an embodiment of the invention;

FIG. 3 shows an overview of a segmentation procedure, in accordance with an embodiment of the invention;

FIG. 4 shows a general CNN architecture used for nervous system structure segmentation, in accordance with an embodiment of the invention;

FIG. 5 shows a flowchart of a training process for the nervous system structure segmentation CNN, in accordance with an embodiment of the invention;

FIG. 6 shows a flowchart of an inference process for the nervous system structure segmentation CNN, in accordance with an embodiment of the invention;

FIG. 7 shows the result of the semantic segmentation of the spine parts and nervous system structures, in accordance with an embodiment of the invention.

FIG. 8 shows the model of the nervous system structures as a result from the segmentation CNN, in accordance with an embodiment of the invention;

FIG. 9A shows the trajectory of a surgical implant colliding with a nervous system structure, in accordance with an embodiment of the invention;

FIG. 9B shows the trajectory of a surgical instrument colliding with a nervous system structure, in accordance with an embodiment of the invention;

FIG. 10 shows a computer-implemented system for implementing the segmentation procedure, in accordance with an embodiment of the invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention.

Several embodiments of the invention relate to processing three dimensional images of nervous system structures in the vicinity of bones, such as nerves of extremities (arms and legs), cervical, thoracic or lumbar plexus, spinal cord (protected by the spinal column), nerves of the peripheral nervous system, cranial nerves, and others. The invention will be presented below based on an example of a spine as a bone in the vicinity of (and at least partially protecting) the nervous system structures, but the method and system can be equally well used for nervous system structures and other bones.

Moreover, the invention may include, before segmentation, pre-processing of low quality images to improve their quality. This can be done by employing a method presented in a European patent application EP16195826 by the present applicant or any other pre-processing quality improvement method. The low quality images may be, for example, low dose computer tomography (LDCT) images or magnetic resonance images captured with a relatively low power scanner

The foregoing description will present examples related to computer tomography (CT) images, but a skilled person will realize how to adapt the embodiments to be applicable to other image types, such as magnetic resonance images.

The nerve structure identification method as presented herein comprises two main procedures in certain embodiments: 1) human-assisted (manual) training, and 2) computer autonomous segmentation.

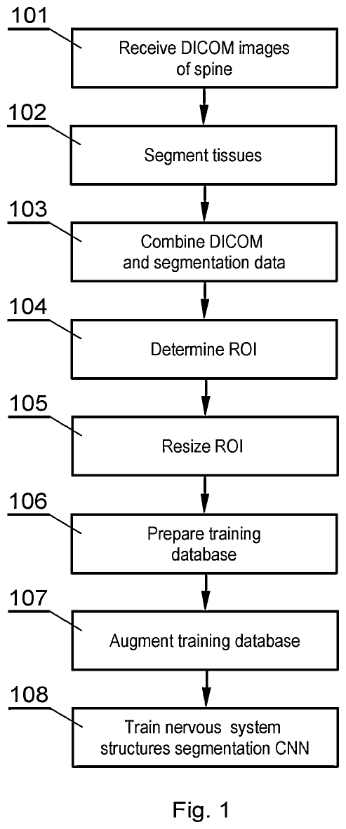

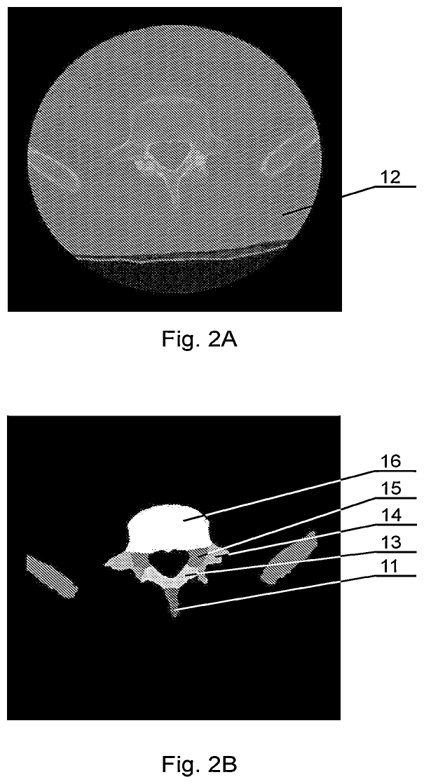

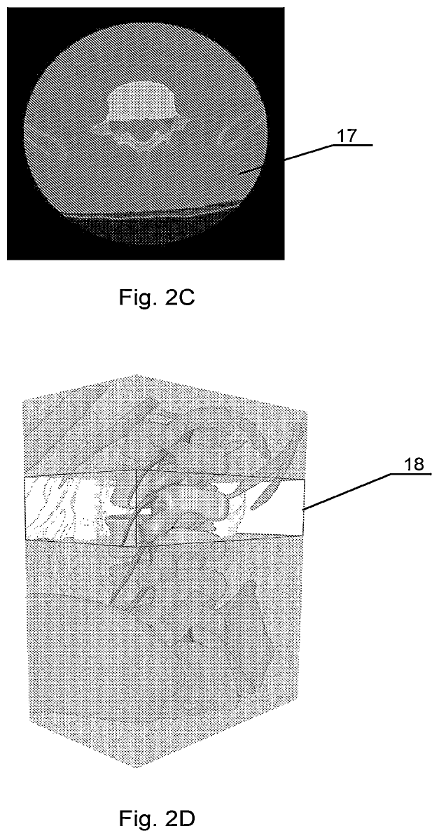

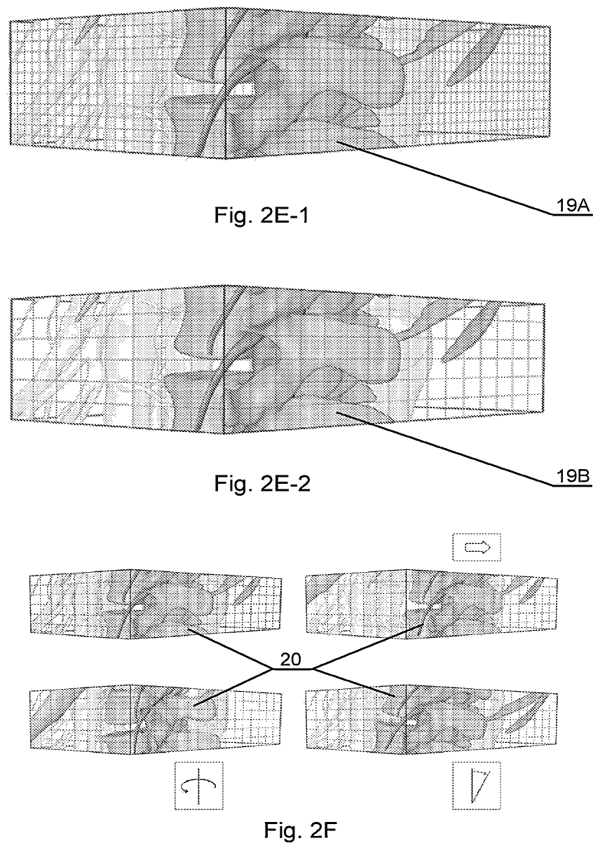

The training procedure, as presented in FIG. 1, comprises the following steps in certain embodiments. First, in step 101, a set of DICOM (Digital Imaging and Communications in Medicine) images obtained with a preoperative or an intraoperative CT or MRI representing consecutive slices of the anatomy, with visible bony and soft tissues (such as one slice 12 shown in FIG. 2A).

Next, the received images are processed in step 102 to perform autonomous segmentation of tissues, in order to determine separate areas corresponding to different parts of the bony structure, such as vertebral body 16, pedicles 15, transverse processes 14 and/or spinous process 11, as shown in FIG. 2B. For example, this can be done by employing a method for segmentation of images disclosed in a European patent application EP16195826 by the present applicant, or any other segmentation method.

Then, in step 103, the information obtained from both original DICOM images and segmentation results is merged to obtain a combined image, comprising information about the tissue appearance and its classification (including assignment of structure parts to classes corresponding to different anatomy parts), for example in a form of a color-coded DICOM image 17, as shown in FIG. 2C. Alternatively, separate DICOM (FIG. 2A) and segmentation (FIG. 2B) images can be processed instead of the combined image.

Next, in step 104, from the set of slice images a 3D region of interest (R01) 18 is determined, that contains, for example, a volume of each vertebral level with a part of surrounding tissues including the nervous system structures and other structures such as muscles, vessels, ligaments, intervertebral discs, joints, cerebrospinal fluid, and others, as shown in FIG. 2D.

Then, in step 105, the 3D resizing of the determined ROI 18 is performed to achieve the same size of all ROI's stacked in the 3D matrices, each containing information about voxel distribution along X, Y and Z axes and the appearance and classification information data of bony structure, such as shown in the resizing (19A) of FIG. 2E-1 and in the resizing (19B) of FIG. 2E-2. In other words, the voxels are small cuboidal volumes resembling points having 3D coordinates and both the original radiodensity value obtained by the scanner and the assigned bony structure classification obtained by the segmentation algorithm

Next, in step 106, a training database is prepared by a human, that comprises the previously determined ROIs and corresponding manually segmented nervous system structures.

Next, in step 107, the training database is augmented, for example with the use of a 3D generic geometrical transformation and resizing with dense 3D grid deformations. An example of such transformation for data augmentation 20 is shown in FIG. 2F. Data augmentation is performed on the images to make the training set more diverse. The foregoing transformations are remapping the voxels positions in a 3D ROI 18 based on a randomly warped artificial grid assigned to the ROI 18 volume. A new set of voxel positions is calculated artificially warping the 3D tissue shape and appearance. Simultaneously, the information about the tissue classification is warped to match the new tissue shape and the manually determined nervous system structure is recalculated in the same manner. During the process, the value of each voxel, containing information about the tissue appearance, is recalculated in regards to its new position in ROI 18 with use of an interpolation algorithm (for example bicubic, polynomial, spline, nearest neighbor, or any other interpolation algorithm) over the 3D voxel neighborhood.

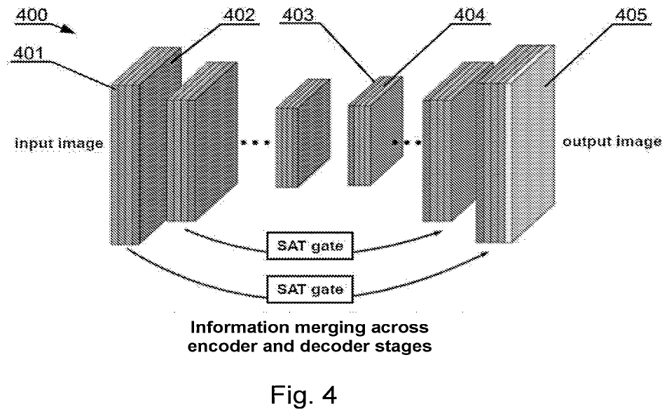

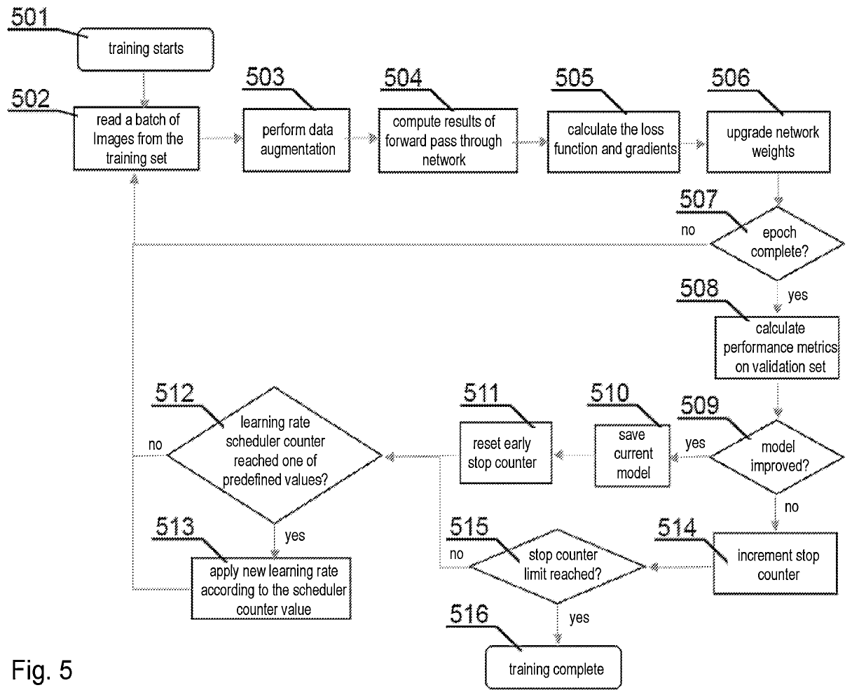

Then, in step 108, a convolutional neural network (CNN) is trained with manually segmented images (by a human) to segment the nervous system structures. In certain embodiments, a network with a plurality of layers can be used, specifically a combination of convolutional with ReLU activation functions or any other non-linear or linear activation functions. For example, a network such as shown in FIG. 4 can be trained according to a process such as shown in FIG. 5. Additionally Select-Attend-Transfer (SAT) gates or Generative Adversarial Networks (GAN) can be used to increase the final quality of the segmentation.

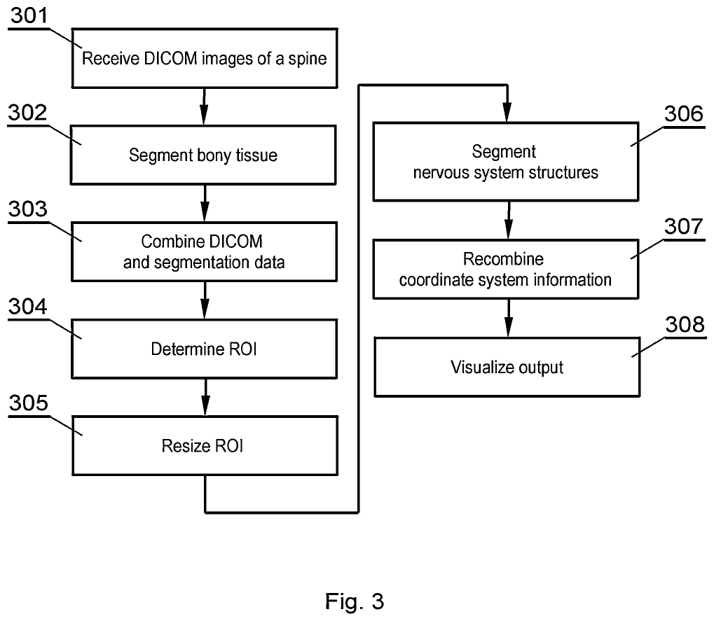

The segmentation procedure, as presented in FIG. 3, comprises the following steps according to certain embodiments. First, in step 301, a 3D scan volume is received, comprising a set of DICOM images of a region of the spinal anatomy. The 3D scan volume can be obtained from a preoperative or an intraoperative CT or MRI. The set of DICOMs representing consecutive slices of the anatomy is received (such as one slice shown in FIG. 2A). Next, the received images are processed in step 302 to perform autonomous segmentation of bony tissues to obtain bony structure segmentation data—such as to determine separate areas corresponding to different spine parts, for example: vertebral body 16, pedicles 15, transverse processes 14, lamina 13 and/or spinous process 11, as shown in FIG. 2B. This step can be done by employing a method for segmentation of images disclosed in a European patent application EP16195826 by the present applicant or any other segmentation method. Then, in step 303, the information obtained from DICOM images and the bony structure segmentation data are merged to obtain combined image comprising information about the tissue appearance and its classification, for example in a form of a color-coded DICOM image, as shown in FIG. 2C. Alternatively, separate DICOM (FIG. 2A) and segmentation (FIG. 2B) images can be processed instead of the combined image. Next, in step 304, a 3D region of interest (ROI) 18 is autonomously determined, which contains a 3D subvolume of the bony structure with a part of surrounding tissues including the nervous system structure and other anatomical components, such as muscles, vessels, ligaments, intervertebral discs, joints, cerebrospinal fluid, and others, as shown in FIG. 2D. Then, in step 305, the 3D resizing of the determined ROI 18 is performed to achieve the same size of all ROI's stacked in the 3D matrices. Each 3D matrix contains information about voxel distribution along X, Y and Z axes with bone density and classification information data for bony structure, such as shown in FIG. 2E. Therefore, steps 301-305 are performed in a way similar to steps 101-105 of the training procedure of FIG. 1.

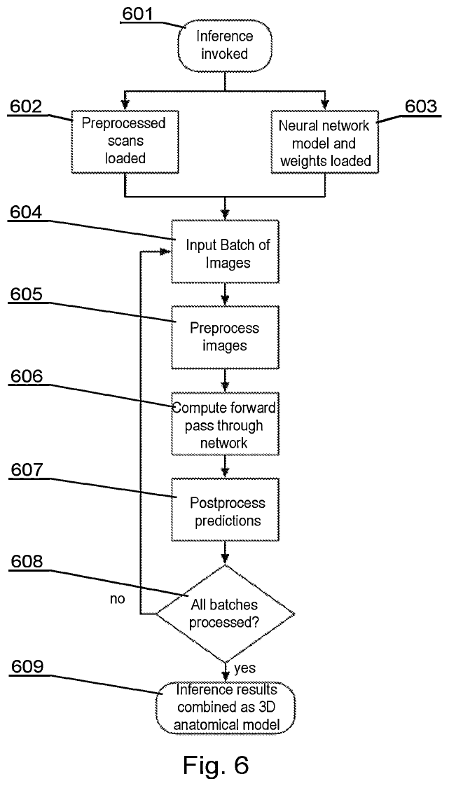

Next, in step 306, the nervous system structures are autonomously segmented by processing the resized ROI to determine the 3D size and shape of the nervous system structure(s), by means of the pretrained nervous-system-structure segmentation CNN 400, as shown in FIG. 4, according to the segmentation process presented in FIG. 6.

In step 307 the information about the global coordinate system (ROI position in the DICOM dataset) and local ROI coordinate system (segmented nervous system structures size, shape and position inside the ROI) is recombined.

Next, in step 308, the output, including the segmented nervous system structures, is visualized.

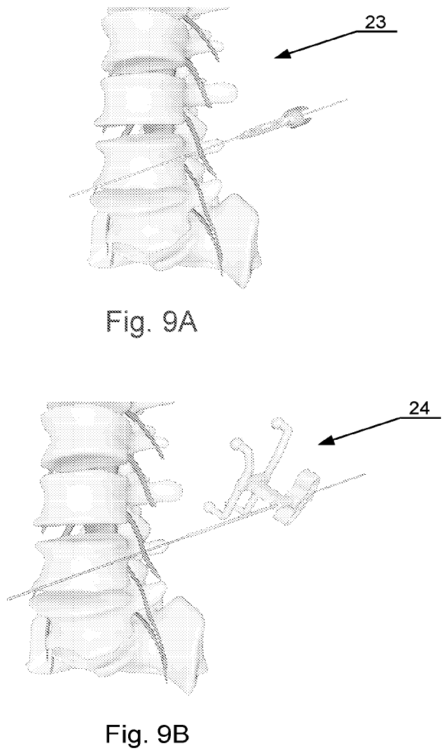

Anatomical knowledge of position, size, and shape of nervous system structure(s) allow for real-time calculation of a possible collision detection with nervous system structure(s) (FIGS. 9A and 9B) while placing medical devices, for example while using a surgical navigation method presented in a European patent application EP18188557.5 by the present applicant. Such collision may result in nervous system structure damage, affecting patient health and quality of life. Autonomous real-time comparison of the position, size, and shape of the nervous system structures, and the medical devices upcoming position, with regard to their size and shape, allow for presenting warnings in the graphical user interface, for example such as presented in a European patent application EP18188557.5 by the present applicant. Moreover, the autonomous collision analysis allows for calculation of change of the preferred medical device position, and can be incorporated, for example, in the method presented in a European patent application EP18188557.5 by the present applicant.

FIG. 4 shows a convolutional neural network (CNN) architecture 400, hereinafter called the nervous-system-structure segmentation CNN, which is utilized in certain embodiments of the method of the invention for both semantic and binary segmentation. The network performs pixel-wise class assignment using an encoder-decoder architecture, using at least one input as a 3D information about the appearance (radiodensity) and the classification of bony structure in a 3D ROI. The left side of the network is a contracting path, which includes convolution layers 401 and pooling layers 402, and the right side is an expanding path, which includes upsampling or transpose convolution layers 403 and convolutional layers 404 and the output layer 405.

One or more 3D ROI's can be presented to the input layer of the network to learn reasoning from the data.

The type of convolution layers 401 can be standard, dilated, or hybrids thereof, with ReLU, leaky ReLU or any other kind of activation function attached.

The type of upsampling or deconvolution layers 403 can also be standard, dilated, or hybrid thereof, with ReLU or leaky ReLU activation function attached.

The output layer 405 denotes the densely connected layer with one or more hidden layer and a softmax or sigmoid stage connected as the output.

The encoding-decoding flow is supplemented with additional skipping connections of layers with corresponding sizes (resolutions), which improves performance through information merging. It enables either the use of max-pooling indices from the corresponding encoder stage to downsample, or learning the deconvolution filters to upsample.

The general CNN architecture can be adapted to consider ROI's of different sizes. The number of layers and number of filters within a layer are also subject to change depending on the anatomical areas to be segmented.

The final layer for binary segmentation recognizes two classes: 1) nervous system structure, and 2) the background).

Additionally Select-Attend-Transfer (SAT) gates or Generative Adversarial Networks (GAN) can be used to increase the final quality of the segmentation. Introducing Select-Attend-Transfer gates to the encoder-decoder neural network results in focusing the network on the most important tissue features and their localization, simultaneously decreasing the memory consumption. Moreover, the Generative Adversarial Networks can be used to produce new artificial training examples.

The semantic segmentation is capable of recognizing multiple classes, each representing a part of the anatomy. For example the nervous system structure may include nerves of the upper and lower extremities, cervical, thoracic or lumbar plexus, the spinal cord, nerves of the peripheral nervous system (e.g., sciatic nerve, median nerve, brachial plexus), cranial nerves, and others.

FIG. 5 shows a flowchart of one embodiment of a training process, which can be used to train the nervous-system-structure segmentation CNN 400. The objective of the training for the segmentation CNN 400 is to tune the parameters of the segmentation CNN 400, so that the network is able to recognize and segment a 3D image (ROI). The training database may be split into a training set used to train the model, a validation set used to quantify the quality of the model, and a test set.

The training starts at 501. At 502, batches of training 3D images (ROIs) are read from the training set, one batch at a time. For the segmentation, 3D images (ROIs) represent the input of the CNN, and the corresponding pre-segmented 3D images (ROIs), which were manually segmented by a human, represent its desired output.

At 503, the original 3D images (ROIs) can be augmented. Data augmentation is performed on these 3D images (ROIs) to make the training set more diverse. The input and output pair of three dimensional images (ROIs) is subjected to the same combination of transformations.

At 504, the original 3D images (ROIs) and the augmented 3D images (ROIs) are then passed through the layers of the CNN in a standard forward pass. The forward pass returns the results, which are then used to calculate at 505 the value of the loss function (i.e., the difference between the desired output and the output computed by the CNN). The difference can be expressed using a similarity metric (e.g., mean squared error, mean average error, categorical cross-entropy, or another metric).

At 506, weights are updated as per the specified optimizer and optimizer learning rate. The loss may be calculated using a per-pixel cross-entropy loss function and the Adam update rule.

The loss is also back-propagated through the network, and the gradients are computed. Based on the gradient values, the network weights are updated. The process, beginning with the 3D images (ROIs) batch read, is repeated continuously until an end of the training session is reached at 506.

Then, at 508, the performance metrics are calculated using a validation dataset—which is not explicitly used in training set. This is done in order to check at 509 whether not the model has improved. If it is not the case, the early stop counter is incremented by one at 514, as long as its value has not reached a predefined maximum number of epochs at 515. The training process continues until there is no further improvement obtained at 516. Then the model is saved at 510 for further use, and the early stop counter is reset at 511. As the final step in a session, learning rate scheduling can be applied. The session at which the rate is to be changed are predefined. Once one of the session numbers is reached at 512, the learning rate is set to one associated with this specific session number at 513.

Once the training process is complete, the network can be used for inference (i.e., utilizing a trained model for autonomous segmentation of new medical images).

FIG. 6 shows a flowchart of an inference process for the nervous-system-structure segmentation CNN 400 according to certain embodiments.

After inference is invoked at 601, a set of scans (three dimensional images) are loaded at 602 and the segmentation CNN 400 and its weights are loaded at 603.

At 604, one batch of three dimensional images (ROIs) at a time is processed by the inference server.

At 605, the images are preprocessed (e.g., normalized, cropped, etc.) using the same parameters that were utilized during training. In at least some implementations, inference-time distortions are applied and the average inference result is taken on, for example, 10 distorted copies of each input 3D image (ROI). This feature creates inference results that are robust to small variations in brightness, contrast, orientation, etc.

At 606, a forward pass through the segmentation CNN 400 is computed.

At 606, the system may perform post-processing such as linear filtering (e.g., Gaussian filtering), or nonlinear filtering (e.g., median filtering, and morphological opening or closing).

At 608, if not all batches have been processed, a new batch is added to the processing pipeline until inference has been performed at all input 3D images (ROIs).

Finally, at 609, the inference results are saved and can be combined into a segmented 3D anatomical model. The model can be further converted to a polygonal mesh for the purpose of visualization. The volume and/or mesh representation parameters can be adjusted in terms of change of color, opacity, changing the mesh decimation depending on the needs of the operator.

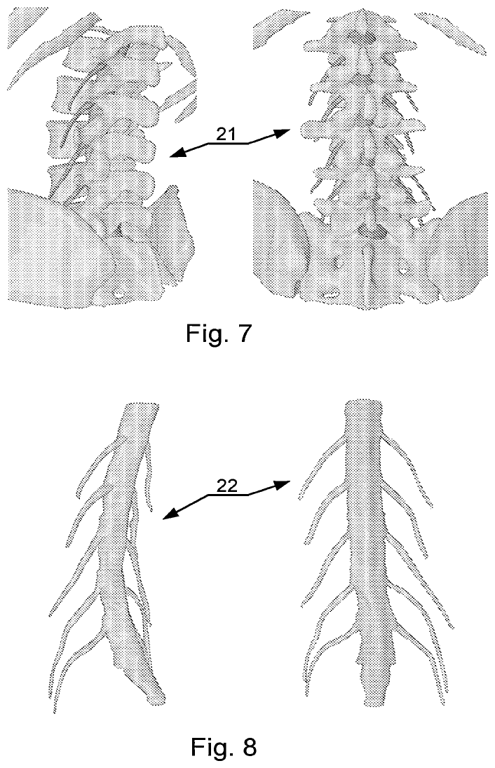

FIG. 7 shows a sample 3D model (21), derived from autonomous segmentation, converted to a polygonal mesh.

FIG. 8 shows a sample 3D model (22), derived from autonomously segmented images presenting a nervous system structure alone.

FIG. 9A shows a sample of the trajectory of a surgical implant (23) colliding with the segmented nervous system structure and FIG. 9B shows the trajectory of a surgical instrument (24) colliding with the segmented nervous system structure.

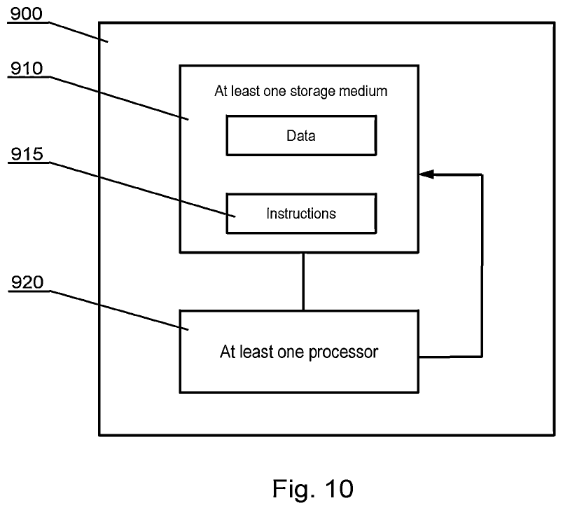

The functionality described herein can be implemented in a computer-implemented system 900, such as shown in FIG. 10. The system may include at least one non-transitory processor-readable storage medium that stores at least one of processor-executable instructions or data and at least one processor communicably coupled to at least one non-transitory processor-readable storage medium. The at least one processor is configured to perform the steps of any particular embodiment of the methods presented herein.

The computer-implemented system 900, for example a machine-learning system, may include at least one non-transitory processor-readable storage medium 910 that stores at least one of processor-executable instructions 915 or data; and at least one processor 920 communicably coupled to the at least one non-transitory processor-readable storage medium 910. The at least one processor 920 may be configured to (by executing the instructions 915) to perform the steps of the method of FIG. 3 in accordance with any embodiment thereof.

While the invention has been described with respect to a limited number of embodiments, it will be appreciated that many variations, modifications and other applications of the invention may be made. Therefore, the claimed invention as recited in the claims that follow is not limited to the embodiments described herein

What is claimed is:

1. A method, comprising:
processing, using a first convolutional neural network (CNN) trained to segment a first type of tissue structure, a set of two-dimensional (2D) images of a three-dimensional (3D) scan volume of a region of patient anatomy to produce segmentation data associated with a set of anatomical parts of the first type within the region of patient anatomy;
generating combined image data by merging the segmentation data associated with the set of anatomical parts of the first type with the set of 2D images;
determining a region of interest (ROI) in the combined image data, the ROI being of a sub-volume of the set of anatomical parts and a neighboring set of anatomical parts of a second type of tissue structure, the ROI including voxels each including data from the set of 2D images and data from the segmentation data associated with the set of anatomical parts; and
processing, using a second CNN trained to segment the second type of tissue structure, the ROI to produce segmentation data associated with the neighboring set of anatomical parts.

2. The method of claim 1, wherein the first type of tissue structure is bony tissue structure, and the second type of tissue structure is nervous system structure.

3. The method of claim 2, wherein:
the set of anatomical parts is of a bony structure,
the data from the set of 2D images includes bone density data of the bony structure, and
the data from the segmentation data includes classification information for the bony structure.

4. The method of claim 2, wherein the set of anatomical parts is a set of spine parts, the set of spine parts being one or more of: a vertebral body, a pedicle, a transverse process, a lamina, or a spinous process.

5. The method of claim 1, further comprising resizing, before processing the ROI using the second CNN, the ROI to have a predefined size suitable for processing using the second CNN, the second CNN having been trained using ROIs having the predefined size.

6. The method of claim 1, further comprising determining a shape, location, and size of the neighboring set of anatomical parts using the segmentation data.

7. The method of claim 6, wherein determining the shape, position, and size of the neighboring set of anatomical parts includes:
determining a shape, position, and size of the neighboring set of anatomical parts in the ROI using the segmentation data; and
combining a local coordinate system of the ROI with a global coordinate system of the 3D scan volume to determine a shape, position, and size of the neighboring set of anatomical parts in the 3D scan volume.

8. The method of claim 6, further comprising:
generating, after determining the shape, location, and size of the neighboring set of anatomical parts, a 3D anatomical model including the neighboring set of anatomical parts; and
displaying, via a display device, a visual representation of the 3D anatomical model.

9. The method of claim 6, further comprising:
detecting, based on the shape, location, and size of the neighboring set of anatomical parts, a possible collision between a medical device and a portion of the neighboring set of anatomical parts; and
displaying, via a display device, a warning of the possible collision.

10. The method of claim 1, further comprising training, before processing the ROI using the second CNN, the second CNN using a training dataset including ROIs having anatomical parts of the first and second types of tissue structure and classification data of the first and second types of tissue structure in each ROI.

11. The method of claim 10, further comprising augmenting the training dataset by:
transforming a set of ROIs using a set of transformations; and
transforming the classification data of the first and second types of tissue structure in each ROI of the set of ROIs using the same set of transformations,
the second CNN being trained using the training dataset after augmenting the training dataset.

12. An apparatus, comprising:
a memory storing instructions; and
a processor operatively coupled to the memory, the processor configured to execute the instructions to:
process, using a first convolutional neural network (CNN) trained to segment a first type of tissue structure, a set of two-dimensional (2D) images of a three-dimensional (3D) scan volume of a region of patient anatomy to produce segmentation data associated with a set of anatomical parts of the first type within the region of patient anatomy;
generate combined image data by merging the segmentation data associated with the set of anatomical parts of the first type with the set of 2D images;
determine a region of interest (ROI) in the combined image data, the ROI being of a sub-volume of the set of anatomical parts and a neighboring set of anatomical parts of a second type of tissue structure, the ROI including voxels each including data from the set of 2D images and data from the segmentation data associated with the set of anatomical parts; and
process, using a second CNN trained to segment the second type of tissue structure, the ROI to produce segmentation data associated with the neighboring set of anatomical parts.

13. The apparatus of claim 12, wherein the processor is further configured to execute the instructions to determine the shape, position, and size of the neighboring set of anatomical parts by:
determining a shape, position, and size of the neighboring set of anatomical parts in the ROI using the segmentation data; and
combining a local coordinate system of the ROI with a global coordinate system of the 3D scan volume to determine a shape, position, and size of the neighboring set of anatomical parts in the 3D scan volume.

14. The apparatus of claim 12, wherein the processor is further configured to execute the instructions to:
determine a shape, location, and size of the neighboring set of anatomical parts using the segmentation data; and
detect, based on the shape, location, and size of the neighboring set of anatomical parts, a possible collision between a medical device and a portion of the neighboring set of anatomical parts.

15. A method, comprising:
receiving a set of two-dimensional (2D) images of a three-dimensional (3D) scan volume of a region of patient anatomy, the set of 2D images including information about tissue appearance of first and second types of tissue structure;
receiving segmentation data including classification information of a set of anatomical parts of the first type of tissue structure within the set of 2D images, the segmentation data obtained using a first convolutional neural network (CNN) trained to segment the first type of tissue structure;
generating combined image data by merging the segmentation data with the set of 2D images, the combined image data including the information about tissue appearance and the classification information of the set of anatomical parts;
determining a region of interest (ROI) in the combined image data, the ROI being of a sub-volume of the set of anatomical parts and a neighboring set of anatomical parts of the second type of tissue structure;
processing, using a second CNN trained to segment the second type of tissue structure, the ROI to produce segmentation data associated with the neighboring set of anatomical parts; and
determining a shape, location, and size of the neighboring set of anatomical parts using the segmentation data associated with the neighboring set of anatomical parts.

16. The method of claim 15, further comprising:
generating a 3D anatomical model including the neighboring set of anatomical parts; and
displaying, via a display device, a visual representation of the 3D anatomical model.

17. The method of claim 15, further comprising:
detecting, based on the shape, location, and size of the neighboring set of anatomical parts, a possible collision between a medical device and a portion of the neighboring set of anatomical parts; and
displaying, via a display device, a warning of the possible collision.

18. The method of claim 15, wherein the combined image data includes a set of color-coded 2D images.

19. The method of claim 15, wherein the first type of tissue structure is bony tissue structure, and the second type of tissue structure is nervous system structure.

20. The method of claim 15, further comprising training, before processing the ROI using the second CNN, the second CNN using a training dataset including ROIs having anatomical parts of the first and second types of tissue structure and classification data of the first and second types of tissue structure in each ROI.
